(19) United States
(12) Patent Application Publication
(10) Pub. No.: US 2006/0264512 A1
(43) Pub. Date: Nov. 23, 2006

(54) Method for the treatment of sexual dysfunction due to medical conditions

(75) Inventor: Robert Pyke, New Fairfield, CT (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/383,796

(22) Filed: May 17, 2006

Related U.S. Application Data
(60) Provisional application No. 60/682,758, filed on May 19, 2005.

(51) Int. Cl. A61K 31/192

(52) U.S. Cl. 514/571

(57) ABSTRACT

The invention relates to a method for the treatment of sexual dysfunctions caused by medical conditions comprising the administration of a therapeutically effective amount of flibanserin.

### RELATED APPLICATIONS

This application claims priority to U.S. Provisional Application Ser. No. 60/682,758, filed on May 19, 2005, the contents of which are incorporated by reference in its entirety.

### FIELD OF THE INVENTION

The invention relates to a method for the treatment of sexual dysfunctions caused by medical conditions comprising the administration of a therapeutically effective amount of flibanserin.

### DESCRIPTION OF THE INVENTION

Several medical conditions like diabetes and hypertension (P. Zemel, American journal of cardiology 61 (16): 27H-33H, 1988), epilepsy (L. Long, Epilepsy & behavior 6 (1): 90-93, 2005), HIV (D. Richardson, HIV Med. 5, Suppl. 2: 21-24, 2004; E. Florence, AIDS care 16 (5): 550-557, 2004), depression, Parkinson's disease etc. are very often associated with sexual dysfunctions

The compound 1-[2-(4-(3-trifluoromethyl-phenyl)piperazin-1-yl)ethyl]-2,3-dihydro-1H-benzimidazol-2-one (flibanserin) is disclosed in form of its hydrochloride in European Patent Application EP-A-526434 and has the following chemical structure:

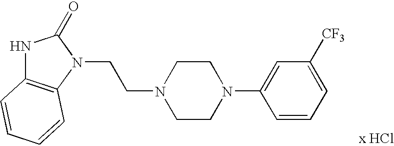

Flibanserin shows affinity for the 5-HT_{1A} and 5-HT₂-receptor. It is therefore a promising therapeutic agent for the treatment of a variety of diseases, for instance depression, schizophrenia and anxiety.

Flibanserin, optionally in form of the free base, the pharmacologically acceptable acid addition salts and/or optionally in form of the hydrates and/or solvates thereof can be used in the treatment of sexual dysfunctions caused by medical conditions.

Therefore, the present invention is directed to a method of treating sexual dysfunctions due to medical conditions comprising administering a therapeutically effective amount of flibanserin, optionally in form of the free base, the pharmacologically acceptable acid addition salts and/or optionally in form of the hydrates and/or solvates thereof to said patient.

As used herein, the term “sexual dysfunction” means a medical diagnosis according to the Diagnostic and Statistical Manual of Mental Disorders, 4th edition, (DSM-IV), Washington D.C., American Psychiatric Association, 1996 and includes the criteria, types, disorders, and subtypes of sexual dysfunction listed therein.

The medical diagnosis of sexual dysfunction is clearly described in the Diagnostic and Statistical Manual of Mental Disorders, 4th edition, (DSM-IV), Washington D.C., American Psychiatric Association, 1996 (incorporated herein by reference). It includes, sexual desire disorders such as hypoactive sexual desire disorder and sexual aversion disorder; sexual arousal disorders such as female sexual arousal disorder and male erectile disorder; orgasmic disorders such as female orgasmic disorder (formerly, inhibited female orgasm), male orgasmic disorder (formerly, inhibited male orgasm), and premature ejaculation; sexual pain disorders such as dyspareunia, noncoital sexual pain disorder and vaginismus. Sexual dysfunction due to medicasl condition are also included in the DSM-IV.

The term “sexual dysfunction due to medical conditions” within the present invention refers to a) sexual desire disorders like female hypoactive sexual desire disorder, male hypoactive sexual desire disorder, female sexual aversion disorder and male sexual aversion disorder all of them caused by medical conditions b) sexual arousal disorders like female sexual arousal disorder and male erectile disorder all of them caused by a medical condition, c) orgasmic disorders such as female orgasmic disorder (formerly, inhibited female orgasm), male orgasmic disorder (formerly, inhibited male orgasm) and premature ejaculation all of them caused by a medical condition as well as d) sexual pain disorders like dyspareunia, noncoital sexual pain disorder and vaginismus all of them caused by a medical condition.

The beneficial effects of flibanserin can be observed regardless of the gender of the patient in need of such treatment.

Accordingly, the instant invention relates to a method for the treatment of sexual dysfunctions caused by medical conditions comprising the administration of a therapeutically effective amount of flibanserin, optionally in form of the free base, the pharmacologically acceptable acid addition salts and/or optionally in form of the hydrates and/or solvates thereof.

In a preferred embodiment the present invention relates to a method for the treatment of sexual dysfunctions caused by medical conditions selected from the group consisting of sexual desire disorders caused by medical conditions, sexual arousal disorders caused by medical conditions, orgasmic disorders caused by medical conditions and sexual pain disorders caused by medical conditions.

In a more preferred embodiment the invention relates to a method for the treatment of sexual desire disorders caused by medical conditions selected from the group consisting of female hypoactive sexual desire disorder (HSDD) caused by medical conditions, male hypoactive sexual desire disorder caused by medical conditions, female sexual aversion disorder caused by medical conditions and male sexual aversion disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of female hypoactive sexual desire disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of male hypoactive sexual desire disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of female sexual aversion disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of male sexual aversion disorder caused by medical conditions, comprising the administration of a therapeutically effective amount of flibanserin, optionally in form of the free base, the pharmacologically acceptable acid addition salts and/or optionally in form of the hydrates and/or solvates thereof.

In another more preferred embodiment the invention relates to a method for the treatment of sexual arousal disorders caused by medical conditions selected from the group consisting of female sexual arousal disorder caused by medical conditions and male erectile disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of female sexual arousal disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of male erectile disorder caused by medical conditions, comprising the administration of a therapeutically effective amount of flibanserin, optionally in form of the free base, the pharmacologically acceptable acid addition salts and/or optionally in form of the hydrates and/or solvates thereof.

In another more preferred embodiment the invention relates to a method for the treatment of orgasmic disorders caused by medical conditions selected from the group consisting of female orgasmic disorder caused by medical conditions, male orgasmic disorder caused by medical conditions and premature ejaculation in male caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of female orgasmic disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of male orgasmic disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of premature ejaculation in male caused by medical conditions, comprising the administration of a therapeutically effective amount of flibanserin, optionally in form of the free base, the pharmacologically acceptable acid addition salts and/or optionally in form of the hydrates and/or solvates thereof.

In a further more preferred embodiment the invention relates to a method for the treatment of sexual pain disorders caused by medical conditions selected from the group consisting of dyspareunia caused by medical conditions, noncoital sexual pain disorder caused by medical conditions and vaginismus caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of dyspareunia caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of noncoital sexual pain disorder caused by medical conditions, comprising the administration of a therapeutically effective amount of flibanserin, optionally in form of the free base, the pharmacologically acceptable acid addition salts and/or optionally in form of the hydrates and/or solvates thereof.

In a even more preferred embodiment the invention relates to a method for the treatment of vaginismus caused by medical conditions, comprising the administration of a therapeutically effective amount of flibanserin, optionally in form of the free base, the pharmacologically acceptable acid addition salts and/or optionally in form of the hydrates and/or solvates thereof.

In a particular preferred embodiment the invention relates to a method for the treatment of female hypoactive sexual desire disorder caused by medical conditions, comprising the administration of a therapeutically effective amount of flibanserin, optionally in form of the free base, the pharmacologically acceptable acid addition salts and/or optionally in form of the hydrates and/or solvates thereof.

Another embodiment of the present invention relates to the use of flibanserin, optionally in form of the free base, the pharmacologically acceptable acid addition salts and/or optionally in form of the hydrates and/or solvates thereof, for the preparation of a medicament for the treatment of the aforementioned dysfunctions.

In another preferred embodiment the invention relates to a method for the treatment of the aforementioned dysfunctions wherein the dysfunction has been caused by a medical condition selected from the group consisting of androgen insufficiency, adrenealectomy, arthritis, chronic fatigue, coronary heart disease, depression, diabetes (type I and II), epilepsy, HIV-infection, hyperprolactinemia, hypogonadism, hypopituitarism, hysterectomy, rectal resection, lower urinary tract symptoms (LUTS) caused by benign prostatic hypertrophy, overactive bladder, stress urinary incontinence, vulvar vestibulitis, interstitial cystitis, multiple sclerosis, oophorectomy, Parkinson's disease, perimenopausal states, postmenopausal states, postpartum states, prostatectomy, radiotherapeutic treatment of cervical cancer, schizophrenia, spinal cord injury, stroke, uraemia, anxiety disorders, somatisation disorder, insomnia, chronic pain syndromes, restless legs syndrome, sleep apnea, chronic forms of hepatitis, irritable bowel syndrome, all forms of cancer including lymphoma and leukemia; myelofibrosis and all forms of anemia and seasonal allergies with systemic disability.

In a preferred embodiment the invention relates to a method for the treatment of the aforementioned dysfunctions wherein the dysfunction has been caused by androgen insufficiency.

In a preferred embodiment the invention relates to a method for the treatment of the aforementioned dysfunctions wherein the dysfunction has been caused by adrenealectomy.

In a preferred embodiment the invention relates to a method for the treatment of the aforementioned dysfunctions wherein the dysfunction has been caused by arthritis.

In a preferred embodiment the invention relates to a method for the treatment of the aforementioned dysfunctions wherein the dysfunction has been caused by chronic fatigue.

In a preferred embodiment the invention relates to a method for the treatment of the aforementioned dysfunctions wherein the dysfunction has been caused by coronary heart disease.

In a preferred embodiment the invention relates to a method for the treatment of the aforementioned dysfunctions wherein the dysfunction has been caused by depression.

In another preferred embodiment the invention relates to a method for the treatment of the aforementioned dysfunctions wherein the dysfunction has been caused by diabetes (type I and II).

In another preferred embodiment the invention relates to a method for the treatment of the aforementioned dysfunctions wherein the dysfunction has been caused by epilepsy.

In another preferred embodiment the invention relates to a method for the treatment of the aforementioned dysfunctions wherein the dysfunction has been caused by HIV-infection.

In another preferred embodiment the invention relates to a method for the treatment of the aforementioned dysfunctions wherein the dysfunction has been caused by hyperprolactinemia.

In another preferred embodiment the invention relates to a method for the treatment of the aforementioned dysfunctions wherein the dysfunction has been caused by hypogonadism.

In another preferred embodiment the invention relates to a method for the treatment of the aforementioned dysfunctions wherein the dysfunction has been caused by hypopituitarism.

In another preferred embodiment the invention relates to a method for the treatment of the aforementioned dysfunctions wherein the dysfunction has been caused by hysterectomy.

In another preferred embodiment the invention relates to a method for the treatment of the aforementioned dysfunctions wherein the dysfunction has been caused by rectal resection.

In another preferred embodiment the invention relates to a method for the treatment of the aforementioned dysfunctions wherein the dysfunction has been caused by lower urinary tract symptoms (LUTS) caused by benign prostatic hypertrophy.

In another preferred embodiment the invention relates to a method for the treatment of the aforementioned dysfunctions wherein the dysfunction has been caused by overactive bladder.

In another preferred embodiment the invention relates to a method for the treatment of the aforementioned dysfunctions wherein the dysfunction has been caused by stress urinary incontinence.

In another preferred embodiment the invention relates to a method for the treatment of the aforementioned dysfunctions wherein the dysfunction has been caused by vulvar vestibulitis.

In another preferred embodiment the invention relates to a method for the treatment of the aforementioned dysfunctions wherein the dysfunction has been caused by interstitial cystitis.

In another preferred embodiment the invention relates to a method for the treatment of the aforementioned dysfunctions wherein the dysfunction has been caused by multiple sclerosis.

In another preferred embodiment the invention relates to a method for the treatment of the aforementioned dysfunctions wherein the dysfunction has been caused by oophorectomy.

In another preferred embodiment the invention relates to a method for the treatment of the aforementioned dysfunctions wherein the dysfunction has been induced by Parkinson's disease.

In another preferred embodiment the invention relates to a method for the treatment of the aforementioned dysfunctions wherein the dysfunction has been caused by perimenopausal states.

In another preferred embodiment the invention relates to a method for the treatment of the aforementioned dysfunctions wherein the dysfunction has been caused by postmenopausal states.

In another preferred embodiment the invention relates to a method for the treatment of the aforementioned dysfunctions wherein the dysfunction has been caused by postpartum states.

In another preferred embodiment the invention relates to a method for the treatment of the aforementioned dysfunctions wherein the dysfunction has been caused by prostatectomy.

In another preferred embodiment the invention relates to a method for the treatment of the aforementioned dysfunctions wherein the dysfunction has been caused by radiotherapeutic treatment of cervical cancer.

In another preferred embodiment the invention relates to a method for the treatment of the aforementioned dysfunctions wherein the dysfunction has been caused by schizophrenia.

In another preferred embodiment the invention relates to a method for the treatment of the aforementioned dysfunctions wherein the dysfunction has been caused by spinal cord injury.

In another preferred embodiment the invention relates to a method for the treatment of the aforementioned dysfunctions wherein the dysfunction has been caused by stroke.

In another preferred embodiment the invention relates to a method for the treatment of the aforementioned dysfunctions wherein the dysfunction has been caused by uraemia.

In another preferred embodiment the invention relates to a method for the treatment of the aforementioned dysfunctions wherein the dysfunction has been caused by anxiety disorders.

In another preferred embodiment the invention relates to a method for the treatment of the aforementioned dysfunctions wherein the dysfunction has been caused by somatisation disorder.

In another preferred embodiment the invention relates to a method for the treatment of the aforementioned dysfunctions wherein the dysfunction has been caused by insomnia.

In another preferred embodiment the invention relates to a method for the treatment of the aforementioned dysfunctions wherein the dysfunction has been caused by chronic pain syndromes.

In another preferred embodiment the invention relates to a method for the treatment of the aforementioned dysfunctions wherein the dysfunction has been caused by restless legs syndrome.

In another preferred embodiment the invention relates to a method for the treatment of the aforementioned dysfunctions wherein the dysfunction has been caused by sleep apnea.

In another preferred embodiment the invention relates to a method for the treatment of the aforementioned dysfunctions wherein the dysfunction has been caused by chronic forms of hepatitis.

In another preferred embodiment the invention relates to a method for the treatment of the aforementioned dysfunctions wherein the dysfunction has been caused by irritable bowel syndrome.

In another preferred embodiment the invention relates to a method for the treatment of the aforementioned dysfunctions wherein the dysfunction has been caused by all forms of cancer including lymphoma and leukemia.

In another preferred embodiment the invention relates to a method for the treatment of the aforementioned dysfunctions wherein the dysfunction has been caused by myelofibrosis and all forms of anemia.

In another preferred embodiment the invention relates to a method for the treatment of the aforementioned dysfunctions wherein the dysfunction has been caused by seasonal allergies with systemic disability.

As already mentioned above, flibanserin may be used in form of the free base, optionally in form of its pharmaceutically acceptable acid addition salts and/or optionally in form of the hydrates and/or solvates thereof. Suitable acid addition salts include for example those of the acids selected from, succinic acid, hydrobromic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularly the hydrochloride, are preferred. If flibanserin is used in form of the free base, it is preferably used in form of flibanserin polymorph A as disclosed in WO 03/014079.

Flibanserin, optionally used in form of its pharmaceutically acceptable acid addition salts and/or optionally in form of the hydrates and/or solvates thereof, or in form of flibanserin polymorph A, may be incorporated into the conventional pharmaceutical preparation in solid, liquid or spray form. The compositions may, for example, be presented in a form suitable for oral, rectal, parenteral administration or for nasal inhalation: preferred forms includes for example, capsules, tablets, coated tablets, ampoules, suppositories and nasal spray.

The active ingredient may be incorporated in excipients or carriers conventionally used in pharmaceutical compositions such as, for example, talc, arabic gum, lactose, gelatine, magnesium stearate, corn starch, acqueous or non acqueous vehicles, polyvynil pyrrolidone, semisynthetic glicerides of fatty acids, benzalconium chloride, sodium phosphate, EDTA, polysorbate 80. The compositions are advantageously formulated in dosage units, each dosage unit being adapted to supply a single dose of the active ingredient. The dosis range of flibanserin applicable per day is between 0.1 to 400, preferably between 1.0 to 300, more preferably between 2 to 200 mg. Each dosage unit may conveniently contain from 0,01 mg to 100 mg, preferably from 0,1 to 50 mg.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g of a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g of. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The Examples which follow illustrate the present invention without restricting its scope:

Examples of pharmaceutical formulations

| | | |
|---|---|---|
| A) | Tablets | per tablet |

| | | |
|---|---|---|
| flibanserin hydrochloride | 100 | mg |
| lactose | 240 | mg |
| corn starch | 340 | mg |
| polyvinylpyrrolidone | 45 | mg |
| magnesium stearate | 15 | mg |
| | 740 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| | | |
|---|---|---|
| B) | Tablets | per tablet |

| | | |
|---|---|---|
| flibanserin hydrochloride | 80 | mg |
| corn starch | 190 | mg |
| lactose | 55 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

C) Coated tablets

| | per coated tablet |
|---|---|
| flibanserin hydrochloride | 5 mg |
| corn starch | 41.5 mg |
| lactose | 30 mg |
| polyvinylpyrrolidone | 3 mg |
| magnesium stearate | 0.5 mg |
| | 80 mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

D) Capsules

| | per capsule |
|---|---|
| flibanserin hydrochloride | 150 mg |
| Corn starch | 268.5 mg |
| Magnesium stearate | 1.5 mg |
| | 420 mg |

The substance and corn starch are mixed and moistened with water. The moist mass is screened and dried. The dry granules are screened and mixed with magnesium stearate. The finished mixture is packed into size 1 hard gelatine capsules.

E) Ampoule solution

| | |
|---|---|
| flibanserin hydrochloride | 50 mg |
| sodium chloride | 50 mg |
| water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

F) Suppositories

| | |
|---|---|
| flibanserin hydrochloride | 50 mg |
| solid fat | 1650 mg |
| | 1700 mg |

The hard fat is melted. At 40° C. the ground active substance is homogeneously dispersed. It is cooled to 38° C. and poured into slightly chilled suppository moulds.

In a particular preferred embodiment of the instant invention, flibanserin is administered in form of specific film coated tablets. Examples of these preferred formulations are listed below. The film coated tablets listed below can be manufactured according to procedures known in the art (see hereto WO 03/097058).

G) Film coated tablet

| Constituents | mg/tablet |
|---|---|
| Core | |
| Flibanserin | 25.000 |
| Lactose monohydrate | 71.720 |
| Microcrystalline cellulose | 23.905 |
| HPMC (Methocel E5) | 1.250 |
| Carboxymethylcellulose sodium | 2.500 |
| Magnesium stearate | 0.625 |
| Coating | |
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 128.000 |

H) Film coated tablet

| Constituents | mg/tablet |
|---|---|
| Core | |
| Flibanserin | 50.000 |
| Lactose monohydrate | 143.440 |
| Microcrystalline cellulose | 47.810 |
| HPMC (e.g. Pharmacoat 606) | 2.500 |
| Carboxymethylcellulose sodium | 5.000 |
| Magnesium stearate | 1.250 |
| Coating | |
| HPMC (e.g. Pharmacoat 606) | 2.400 |
| Polyethylene Glycol 6000 | 0.700 |
| Titanium dioxide | 1.000 |
| Talc | 0.857 |
| Iron oxide red | 0.043 |
| Total Film coated tablet | 255.000 |

I) Film coated tablet

| Constituents | mg/tablet |
|---|---|
| Core | |
| Flibanserin | 100.000 |
| Lactose monohydrate | 171.080 |
| Microcrystalline cellulose | 57.020 |
| HPMC (e.g. Methocel E5) | 3.400 |
| Carboxymethylcellulose sodium | 6.800 |
| Magnesium stearate | 1.700 |
| Coating | |
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated tablet | 347.000 |

J) Film coated tablet

| Constituents | mg/tablet |
|---|---|
| Core | |
| Flibanserin | 2.000 |
| Dibasic Calciumphosphate, anhydrous | 61.010 |
| Microcrystalline cellulose | 61.010 |
| HPMC (Methocel E5) | 1.950 |
| Carboxymethylcellulose sodium | 2.600 |
| Colloidal silicon dioxide | 0.650 |
| Magnesium stearate | 0.780 |
| Coating | |
| HPMC (Methocel E5) | 1.440 |
| Polyethylene Glycol 6000 | 0.420 |
| Titanium dioxide | 0.600 |
| Talc | 0.514 |
| Iron oxide red | 0.026 |
| Total Film coated tablet | 133.000 |

K) Film coated tablet

| Constituents | mg/tablet |
|---|---|
| Core | |
| Flibanserin | 100.000 |
| Dibasic Calciumphosphate, anhydrous | 69.750 |
| Microcrystalline cellulose | 69.750 |
| HPMC (e.g. Methocel E5) | 2.750 |
| Carboxymethylcellulose sodium | 5.000 |
| Colloidal silicon dioxide | 1.250 |
| Magnesium stearate | 1.500 |
| Coating | |
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 255.000 |

L) Film coated tablet

| Constituents | mg/tablet |
|---|---|
| Core | |
| Flibanserin | 20.000 |
| Lactose monohydrate | 130.000 |
| Microcrystalline cellulose | 43.100 |
| Hydroxypropyl Cellulose (e.g. Klucel LF) | 1.900 |
| Sodium Starch Glycolate | 4.000 |
| Magnesium stearate | 1.000 |
| Coating | |
| HPMC (e.g. Methocel E5) | 2.400 |
| Polyethylene Glycol 6000 | 0.700 |
| Titanium dioxide | 1.043 |
| Talc | 0.857 |
| Total Film coated tablet | 205.000 |

I claim:

1) A method for the treatment of a sexual dysfunction caused by a medical condition comprising the administration of a therapeutically effective amount of flibanserin, or a pharmacologically acceptable acid addition salt thereof, or a hydrate or a solvate thereof.

2) The method for the treatment of a sexual dysfunction caused by a medical condition according to claim 1, wherein the sexual dysfunction is selected from the group consisting of sexual desire disorders caused by a medical condition, sexual arousal disorders caused by a medical condition, orgasmic disorders caused by a medical condition, sexual pain disorders caused by a medical condition and combinations thereof.

3) The method according to claim 1, wherein the sexual dysfunction is a sexual desire disorder caused by a medical condition.

4) The method according to claim 1, wherein the sexual dysfunction is a sexual arousal disorder caused by a medical condition.

5) The method according to claim 1, wherein the sexual dysfunction is an orgasmic disorder caused by a medical condition.

6) The method according to claim 1, wherein the sexual dysfunction is a sexual pain disorder caused by a medical condition.

7) The method according to claim 1, wherein the sexual dysfunction has been caused by a medical condition selected from the group consisting of androgen insufficiency, adrenealectomy, arthritis, chronic fatigue, coronary heart disease, depression, diabetes (type 1 and 11), epilepsy, HIV-infection, hyperprolactinemia, hypogonadism, hypopituitarism, hysterectomy, rectal resection, lower urinary tract symptoms (LUTS) caused by benign prostatic hypertrophy, overactive bladder, stress urinary incontinence, vulvar vestibulitis, interstitial cystitis, multiple sclerosis, oophorectomy, Parkinson's disease, perimenopausal states, postmenopausal states, postpartum states, prostatectomy, radiotherapeutic treatment of cervical cancer, schizophrenia, spinal cord injury, stroke, uraemia, anxiety disorders, somatisation disorder, insomnia, chronic pain syndromes, restless legs syndrome, sleep apnea, chronic forms of hepatitis, irritable bowel syndrome, any form of cance, myelofibrosis, any form of anemia and seasonal allergies with systemic disability.

8) The method according claim 1, wherein flibanserin is in the form of a pharmaceutically acceptable acid addition salt, wherein the pharmaceutically acceptable acid addition salt is formed by an acid selected from the group consisting of succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

9) The method according to claim 1, wherein flibanserin is in the form of flibanserin polymorph A.
